(19) United States
(12) Patent Application Publication
(10) Pub. No.: US 2002/0165501 A1
(43) Pub. Date: Nov. 7, 2002

(54) Safety Syringe

(76) Inventor: Zhan Bo Yang, Taipei (TW)

Correspondence Address: ZHAN BO YANG, P.O. BOX 26-757, TAIPEI, TW

(21) Appl. No.: 09/850,593

(22) Filed: May 4, 2001

(51) Int. Cl. A61M 5/31

(52) U.S. Cl. 604/240; 604/195

(57) ABSTRACT

A safety syringe comprises a syringe body spaced by a spacer to be formed with a first receiving chamber and a second receiving chamber; and the spacer being installed with a first through hole; a control unit slidably matched to the second receiving chamber; and a front end of the control unit being installed with a second through hole; the base being connected to a front end of the control unit; and a push rod slidably moving in the first receiving chamber of the syringe body. Thereby, the syringe can be received in the second receiving chamber. When injection, the control unit and syringe are pushed to the second receiving chamber for moving forwards. After injection, the control unit is pushed so that the syringe is pulled into the second receiving chamber of the syringe body so as to prevent from hurting anyone.

### FIELD OF THE INVENTION

The present invention relates to a safety syringe which can prevent from hurting anyone effectively, and thus has a high safety quality, moreover, the safety syringe can be manufactured easily and therefore, has a lower cost.

### BACKGROUND OF THE INVENTION

The prior art syringe is formed by a syringe body, a syringe needle, and a push rod. The front end of the syringe needle has a tip portion so that it is easy to prick somebody. Especially, a used syringe needle will possible infect disease to those pricked by the syringe needle. Therefore, safety syringes are developed, such as those disclosed in Taiwan Patent No. 312154, 320057, 349405, 357615, 395247, etc. In these patents, various kinds of safety syringe are disclosed. In most of these structures, a buckling structure is added between the front end of the push rod and a base at the rear end of the syringe needle. Therefore, after injection, the push rod can be combined with the base at the rear end of the syringe needle. Therefore, as the syringe needle is pulled into the syringe body, the syringe needle is prevented from prick anyone.

However, the prior art safety syringes are only used to prevent from pricking anyone since after the safety syringe is used, the syringe needle is pulled back. Before injection, the syringe needle is exposed out and therefore, the packing and transferring process, the syringe needle is possible to prick somebody.

Furthermore, in the prior art safety syringe, after injection, the push rod must be buckled to the syringe needle for pulling the syringe needle into the syringe body. Therefore, a high precision is required to the buckling structure between the front end of the push rod and the base in the rear end.

### SUMMARY OF THE INVENTION

Accordingly, the primary object of the present invention is to provide a safety syringe, wherein the syringe can be received in the second receiving chamber. When injection, the control unit and syringe are pushed to the second receiving chamber for moving forwards. After injection, the control unit is pushed so that the syringe is pulled into the second receiving chamber of the syringe body so as to prevent from hurting anyone. Thus the control unit of the present invention has a high safety quality.

Another object of the present invention is to provide a safety syringe, which can be manufactured easily and thus has a lower cost.

To achieve the object, the present invention provides a safety syringe comprising a syringe body spacing by a spacer to be formed with a first receiving chamber and a second receiving chamber; a front end of the second receiving chamber being formed as an opening; and the spacer being installed with a first through hole; a control unit slidably matched to the second receiving chamber; a front end of the control unit being installed with a second through hole; and another end of the second through hole penetrating through one lateral wall of the control unit; and a syringe needle having a rear end connected to a base; the base being connected to a front end of the control unit; wherein the syringe needle and control unit cooperates in the second receiving chamber of the syringe body; and a push rod slidably moving in the first receiving chamber of the syringe body.

The various objects and advantages of the present invention will be more readily understood from the following detailed description when read in conjunction with the appended drawing.

### BRIEF DESCRIPTION OF THE DRAWINGS

FIG. 1 is an assembled perspective view of the first embodiment in the present invention.

FIG. 2 is an exploded perspective view of the first embodiment in the present invention.

FIG. 3 is a cross sectional view of the first embodiment in the present invention.

FIG. 4 is the cross sectional view showing the syringe being pushed out of the first embodiment in the present invention.

FIG. 5 is a cross sectional view showing liquid is absorbed out that of the first embodiment in the present invention.

FIG. 6 is a cross sectional view showing the second embodiment of the present invention.

FIG. 7 is a perspective view of the control unit in the third embodiment of the present invention.

FIG. 8 is a cross sectional view of the third embodiment in the present invention.

FIG. 9 is a schematic view showing the action of the third embodiment in the present invention.

FIG. 10 is a cross sectional view of the fourth embodiment in the present invention.

FIG. 11 is a cross sectional view showing that the syringe head is pushed out of the fourth embodiment in the present invention.

### DETAILED DESCRIPTION OF THE PREFERRED EMBODIMENTS

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

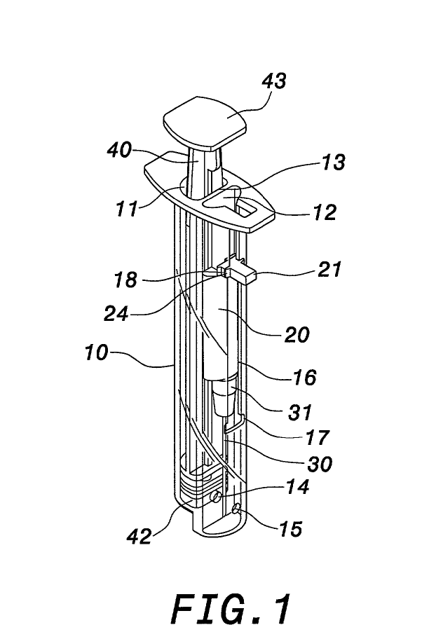

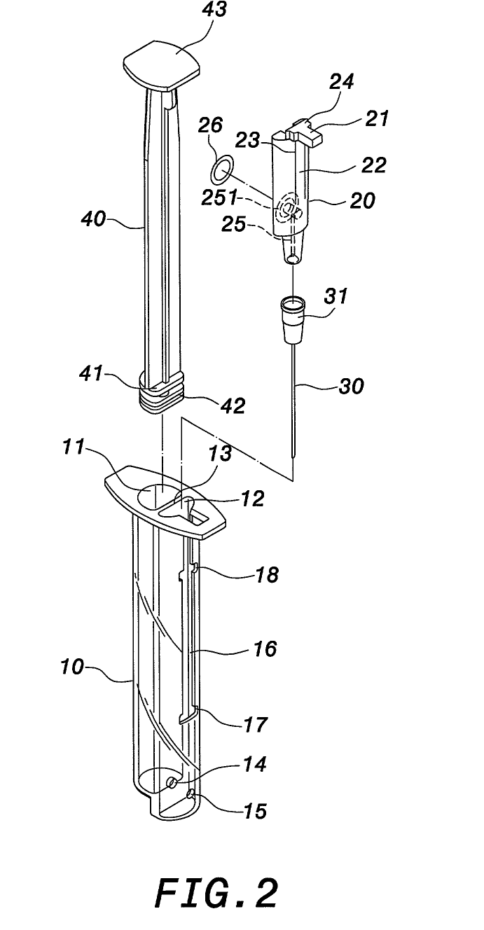

Referring to FIGS. 1, 2 and 3, the assembled perspective view, exploded perspective view and cross sectional view of the present invention are illustrated. The safety syringe of the present invention includes a syringe body 10, a control unit 20, a syringe needle 30, and a push rod 40. The syringe body 10 is a hollow tube with a spacer 13 for spacing the interior thereof into a first receiving chamber 11 and a second receiving chamber 12. The rear ends of the first receiving chamber 11 and second receiving chamber 12 are opened. The cross section of the first receiving chamber 11 is larger than the cross section of the second receiving chamber 12. The front end of the second receiving chamber 12 is opened and is protruded from the front end of the first receiving chamber

The upper front end of the spacer 13 between the first receiving chamber 11 and second receiving chamber 12 are installed with a first through hole 14. A penetrating hole 15 is installed at one lateral wall of the second receiving chamber 12. The penetrating hole 15 is correspondent to the first through hole 14. The installation of the penetrating hole 15 is beneficial for forming the first through hole 14 externally so as to be formed as a channel for flowing the syringe liquid. The lateral wall of the second receiving chamber 12 has a sliding groove 16 extending along the length. A front positioning hole 17 and a rear positioning hole 18 are formed in the sliding groove 16.

The control unit 20 is shorter than that of the syringe body 10 and is like a long rod correspondent to the second receiving chamber 12. The control unit 20 is slidably matched to the second receiving chamber 12. A push button 21 is installed in the control unit 20. The front end of the push button 21 is connected to an elastic arm 22. The elastic arm 22 is formed on the control unit 20. Between two sides of the elastic arm 22 and control unit 20 are installed with trenches 23. Therefore, a free end is formed at the rear end of the elastic arm 22 so that the push button 21 connected to the rear end of the elastic arm 22 may elastically move inwards or outwards. The inner side of the push button 21 is installed with a positioning body 24.

The push button 21 of the control unit 20 is engaged to the sliding groove 16 of the syringe body 10. When the push button 21 is pressed inwards, the positioning body 24 will separate from the front positioning hole 17 or rear positioning hole 18. The control unit 20 can be driven by pushing the push button 21 to move forwards or rearwards in the second receiving chamber 12. When the push button 21 moves to the front maximum or rear maximum, the force pressed the push button 21 inwards can be released so that the positioning body 24 of the push button 21 is buckled and positioned to the front end of the sliding groove 16 or the front positioning hole 17 or rear positioning hole 18 at the rear end.

The front end of the control unit 20 is installed with a second through hole 25. The rear end of the second through hole 25 extends and penetrates through the lateral wall of the control unit 20 to be communicated with external environment. The periphery of the rear end of the second through hole 25 is installed with an annular groove 251. The annular groove 251 has a matched sealing ring 26 for enhancing the tightness between the control unit 20 and the second receiving chamber 12.

The syringe needle 30 is a slender hollow tube, and the front end is tip portion. The rear end thereof is connected to a hollow base 31 which is engaged with the front end of the control unit 20 so that the syringe and the control unit 20 are matched to the second receiving chamber 12 of the syringe body 10.

The push rod 40 is a long rod with a front end forming with a head 41. The head 41 is engaged with a piston 42. The shape of the piston 42 is correspondent to the first receiving chamber 11 of the syringe body 10. The rear end of the push rod 40 is formed with a larger plate pushing portion 43. The push rod 40 is slidably matched to the first receiving chamber 11 of the syringe body 10. The piston 42 is tightly engaged with the first receiving chamber 11 so that a proper tightness is retained between the piston 42 and the first receiving chamber 11. The pushing portion 43 pushes the push rod 40 to move forwards and rearwards in the first receiving chamber 11. By above assembly, the safety syringe of the present invention is formed.

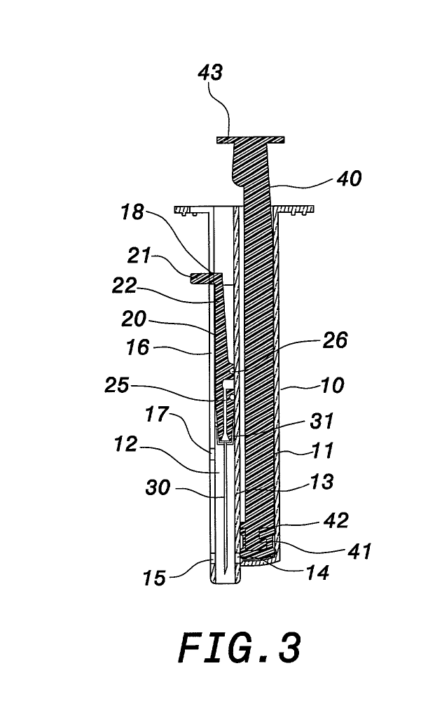

Referring to FIG. 3, the syringe of the present invention is received in the second receiving chamber 12 of the syringe body 10 before injection. The position body 24 of the push button 21 of the control unit 20 is positioned in the rear positioning hole 18 at the rear end of the sliding groove 16. The position body 24 of the push button 21 is engaged with the rear positioning hole 18 to be formed as a first positioning unit. The syringe needle 30 of the control unit 20 is positioned in the second receiving chamber 12 of the syringe body 10. Therefore, before injection, the syringe needle 30 will not expose out. Therefore, in the process of packaging or transferred the syringe, it can prevent the syringe to hurt others.

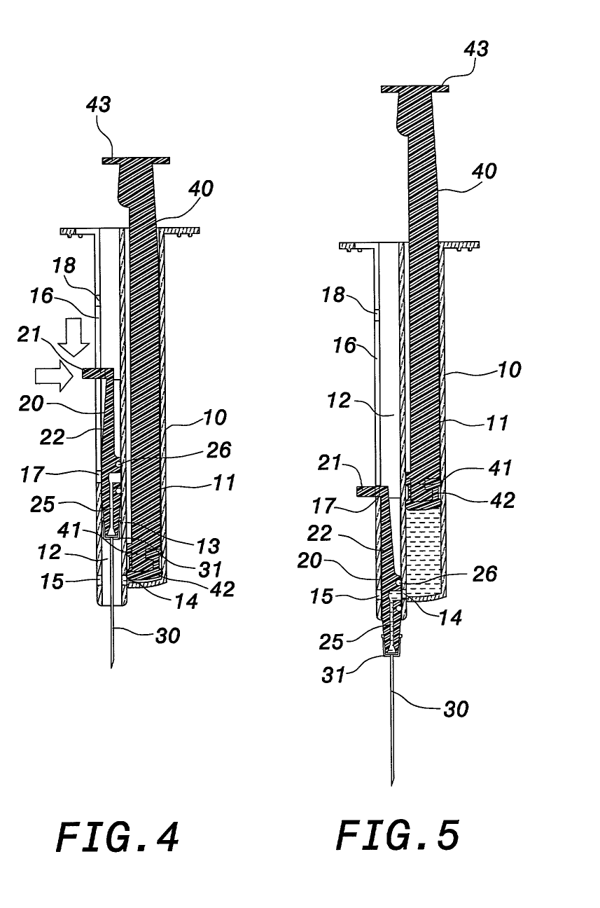

Referring to FIG. 4, as the syringe needle 30 is used, the push button 21 is pressed inwards so that the position body 24 of the push button 21 is separated from the rear positioning hole 18. Then, the push button 21 will push the control unit 20 to move forwards in the second receiving chamber 12. When the push button 21 is pushed to the front-most, the force inwards pressing the push button 21 can be released so that the position body 24 of the push button 21 is engaged and positioned to the front positioning hole 17 at the front end of the sliding groove 16 so that the position body 24 of the push button 21 is buckled to the front positioning hole 17 to be formed as a second positioning unit. The control unit 20 is positioned in the front end of the second receiving chamber 12 of the syringe body 10, and the front end of the syringe needle 30 protrudes out of the front end of the second receiving chamber 12 of the syringe body 10 (FIG. 5). Then, the second through hole 25 of the control unit 20 is at a position with respect to the first through hole 14 of the syringe body 10 to form a free flowing channel between the first through hole 14 and second through hole 25.

With reference to FIG. 5, the push portion 43 pushes the push rod 40 to move backwards in the first receiving chamber 11, and then the syringe needle 30, second through hole 25 of the control unit 20, and first through hole 14 of the syringe body 10 pumps the injection liquid to enter into the first receiving chamber 11 so that the push portion 43 push the push rod 40 to move forwards in the first receiving chamber 11. Through the first through hole 14 of the syringe body 10, second through hole 25 of the control unit 20, and syringe needle 30, the injection liquid is injected to human body.

Referring to FIG. 3, after injection by the syringe needle 30, pressing the push button 21 inwards, the position body 24 of the push button 21 will separate from the front positioning hole 17, then the push button 21 will push the control unit 20 to move backwards in the second receiving chamber 12. When the push button 21 is pushed backwards to the rear most, the force inwards pressing the push button 21 can be released so that the position body 24 of the push button 21 is buckled and positioned in the rear positioning hole 18 at the rear end of the sliding groove 16. Positioning the control unit 20 in the second receiving chamber 12 of the syringe body 10 cause the syringe needle 30 is pulled into the syringe body 10 after injection so as to prevent the syringe needle 30 from hurting anyone.

In the safety syringe of the present invention, the syringe needle 30 can be pulled into the syringe body 10 to prevent the syringe needle 30 to hurt anyone. Before injection, the syringe needle 30 can be received in the second receiving chamber 12 of the syringe body 10, therefore, in the packing and transferring process of the syringe, the syringe is prevented from hurting anyone. Furthermore, in the present invention, by the push button 21 of the control unit 20 to push the syringe needle 30 to expose out or reduce inwards, no high precision buckling structure is necessary to be installed between the rear base of the syringe needle 30 of the push rod 40 and thus, the manufacturing process is easy and the cost is low.

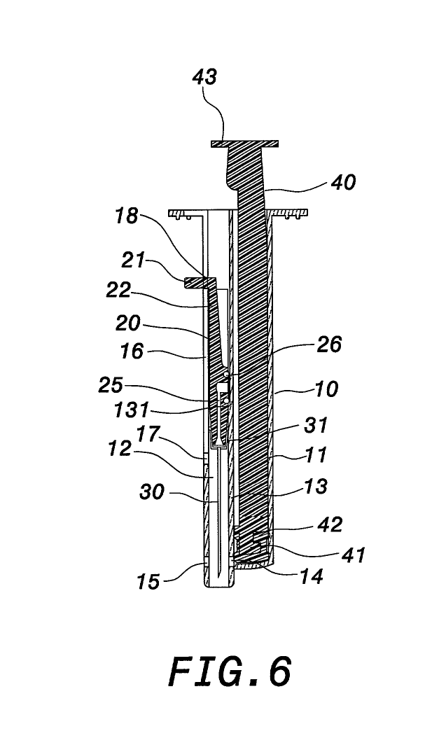

Referring to FIG. 6, the cross sectional view of the second embodiment of the present invention is illustrated, a tilt surface 131 is installed at the surface of the spacer 13 of the first receiving chamber 11 and second receiving chamber 12, wherein the surface is connected to the second receiving chamber 12. Therefore, when the control unit 20 is pushed to move forwards in the second receiving chamber 12, the tilt surface 131 will press the sealing ring 26 gradually for enhancing the tightness between the control unit 20 and the second receiving chamber 12.

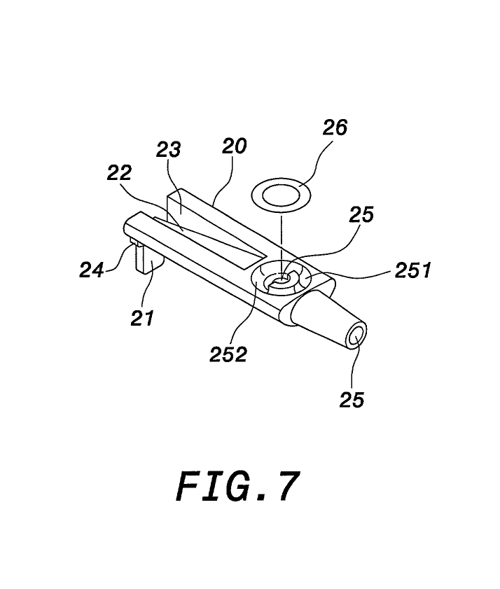

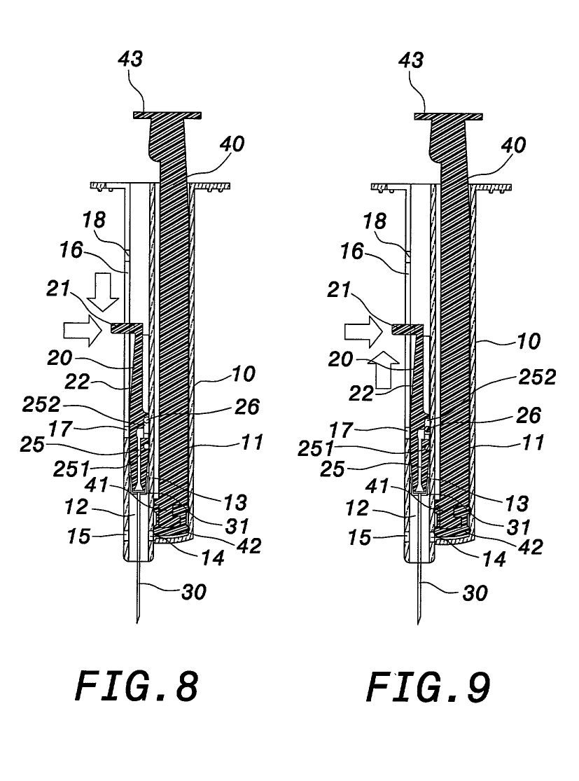

Referring to FIG. 8, the cross sectional view of the third embodiment according to the present invention is illustrated. The rear half of the annular groove 251 at the outer periphery of the rear end of the second receiving chamber 12 in the control unit 20 is formed with a recess 252 (referring to FIG. 7), and thus, when pushing the control unit 20 to displace forwards in the second receiving chamber 12, the front half of the sealing ring 26 is positioned to the annular groove 251. The rear half of the sealing ring 26 is positioned to the edge of the recess 251 so that the sealing ring 26 may work normally (referring to FIG. 8) to increase the tightness between the control unit 20 and the second receiving chamber 12.

After the syringe needle 30 is used, when pushing the control unit 20 to move rearwards in the second receiving chamber 12, the rear half of the sealing ring 26 contacts and rubber with the spacer 13 so as to be pushed by the spacer 13 to separate from the edge of the recess 252 (referring to FIG. 9). As a result, the sealing ring 26 will deform and destroy not to work normally. Therefore, the syringe can not be reused repeatedly, thereby the safety of the syringe is increased.

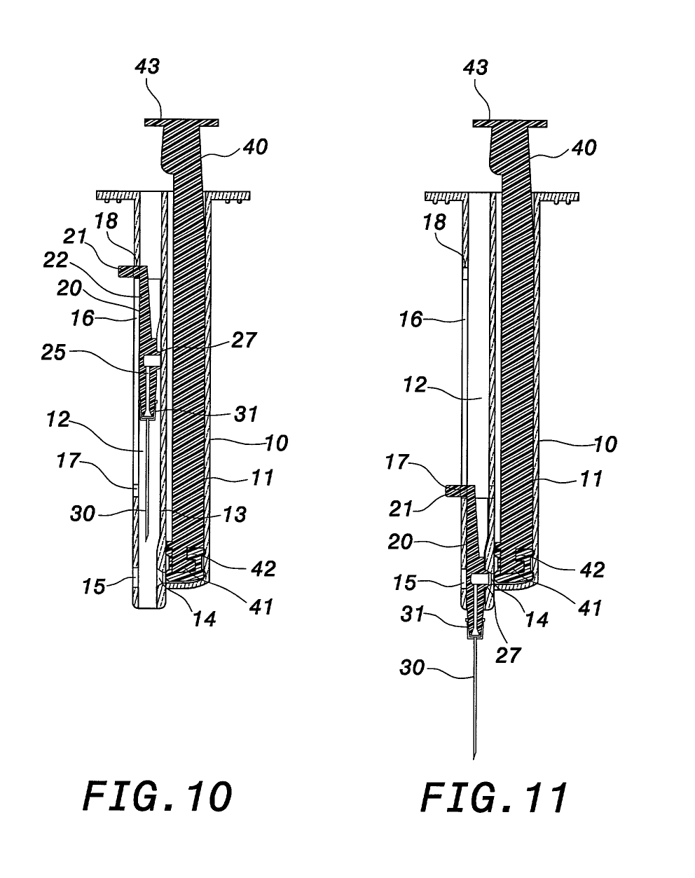

Furthermore, referring to FIGS. 10 and 11, in the present invention, a protrusion 27 can be added at the outer periphery of the rear end of the second though hole 25 of the control unit 20 and the first though hole 14 is designed to have a taper shape. Therefore, as the control unit 20 is pushed to displace forwards in the second receiving chamber 12, the protrusion 27 can be embedded into the first though hole 14 for enhancing the sealing between the control unit 20 and the second receiving chamber 12.

In summary, the present invention has improved the defects in the prior art, such as hurting to people, high cost, etc. Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A safety syringe, comprising:
a syringe body spaced by a spacer so as to form with a first receiving chamber and a second receiving chamber; a front end of the second receiving chamber being formed as an opening; and the spacer being installed with a first through hole;
a control unit slidably matched to the second receiving chamber; a front end of the control unit being installed with a second through hole; and another end of the second through hole penetrating through one lateral wall of the control unit; and
a syringe needle having a rear end connected to a base; the base being connected to a front end of the control unit; wherein the syringe needle and control unit cooperates in the second receiving chamber of the syringe body; and
a push rod slidably moving in the first receiving chamber of the syringe body.

2. The safety syringe as claimed in claim 1, wherein rear ends of the first receiving chamber and second receiving chamber of the syringe body are formed with openings.

3. The safety syringe as claimed in claim 1, wherein a front end of second receiving chamber of the syringe protrudes out of a front end of the first receiving chamber.

4. The safety syringe as claimed in claim 1, wherein one lateral wall of the second receiving chamber is installed with a positioning hole positioned with respect to the first through hole.

5. The safety syringe as claimed in claim 1, wherein the first through hole of the syringe body is installed at a front end of the spacer.

6. The safety syringe as claimed in claim 1, wherein a lateral wall of the second receiving chamber has a sliding groove extending along the length direction; a front positioning hole and a rear positioning hole are formed in the sliding groove; a push button is installed in the control unit; an inner side of the push button is installed with a positioning body; the push button of the control unit is matched in the sliding groove of the syringe body; the position body of the push button is buckled to the rear positioning hole to be formed as a first unit; and the position body of the push button is buckled to the front positioning hole to be formed as a second positioning unit.

7. The safety syringe as claimed in claim 1, wherein an annular groove is formed at a periphery of a rear end of the second receiving chamber in the control unit.

8. The safety syringe as claimed in claim 7, wherein a surface of the spacer connected to the spacer is a tilt surface so that the thickness of the spacer is forward-increased; when the control unit moves forwards in the second receiving chamber, the tilt surface will tighten the sealing ring gradually.

9. The safety syringe as claimed in claim 7, wherein a recess is formed at rear half of the annular groove of the control unit; when the control unit is pushed to move in the second receiving chamber, a rear half of the sealing ring is positioned in an edge of the recess; when the control unit is pushed to move backwards in the second receiving chamber, the rear half of the sealing ring separates from the edge of the recess so that the sealing ring is deformed and destroyed.

10. The safety syringe as claimed in claim 1, wherein a protrusion is formed at an outer periphery of a rear end of the second though hole of the control unit and the first though hole is designed to have a taper shape, wherein the protrusion is embedded into the first though hole.

11. The safety syringe as claimed in claim 1, wherein a front end of the push rod has a head, and a plug is engaged to the head.

12. The safety syringe as claimed in claim 1, wherein a rear end of the push rod is formed with a large plate push portion.
